(19) United States
(12) Patent Application Publication
(10) Pub. No.: US 2006/0083700 A1
(43) Pub. Date: Apr. 20, 2006

(54) Use of a novel phytonutrient rich bioactive concentrate (Ri-ActiveTM) for the prevention and treatment of cardiovascular disease, diabetes and other health disorders

(75) Inventors: Reddy Sastry V. Cherukuri, Folsom, CA (US); Rukmini C. Cherukuri, Folsom, CA (US); Aravind Cherukuri, Newton, MA (US); Kartik Natarajan, West Hartford, CT (US)

Correspondence Address: VENABLE LLP, P.O. BOX 34385, WASHINGTON, DC, 20045-9998, US

(73) Assignee: NaturRi LLC, West Hartford, CT (US)

(21) Appl. No.: 11/251,875

(22) Filed: Oct. 18, 2005

Related U.S. Application Data
(60) Provisional application No. 60/619,879, filed on Oct. 18, 2004.

(51) Int. Cl. A61K 36/899; A61K 47/00; A61K 8/97

(52) U.S. Cl. 424/59; 424/750; 424/439

(57) ABSTRACT

The present invention is generally related to the discovery of the use of a novel composition (Ri-Active™), containing a concentrate of the bioactives, micronutrients and antioxidants present in the unsaponifiable fraction of rice bran oil or rice germ oil, in the prevention and treatment of health disorders. The novel use of the by-products of rice bran oil or rice germ (and other vegetable oils) industries directly after purification and stabilization for therapeutic purposes is a unique and innovative approach that has been proved in the pilot study conducted by the current inventors. The current inventors have pioneered a new approach to prevent and treat health disorders such as cardiovascular diseases, hypercholesterolemia and diabetes which is both effective and inexpensive. The proprietary technology developed by the current inventors to produce Ri-Active™ from rice bran oil by-products or rice germ is very different from others who use these by-products to either isolate or purify individual micronutrients of value for use in other nutraceutical formulations. Most of the by-products of the edible oil refining industry are either discarded or sold at a throw away cost to the soap industry for their free fatty acid content. The novel technology and approach developed by the current inventors to utilize these low cost by-products from the edible oil refining industry and produce a highly effective therapeutic product has yielded a new and valuable use for these materials. This invention extends to the uses (therapeutic usefulness) and cost effectivity of similar whole extract concentrates of unsaponifiable micronutrients prepared from other vegetable oils rich in unsaponifiable fractions. The current discovery specifically covers the uses of Ri-Active™ (derived either from rice bran oil or rice germ oil), in preventing or treating cardiovascular disease, diabetes, hyperlipidemia, hypercholesterolemia and arteriosclerosis. Ri-Active™ contains many unsaponifiable bioactives and antioxidants of rice bran and rice germ in very high concentrations in a natural lipid matrix. These constituents in their natural lipid matrix act synergistically in the treatment of cardiovascular disease and other disorders and therefore the whole extract concentrate containing these constituents is far more efficacious than administering a mixture of procured ingredients in similar concentrations. The efficacy of this novel composition (Ri-Active™) in the treatment of cardiovascular disease risk factors has been proved in the pilot animal study conducted by the current inventors. Ri-Active™ has been tested to be safe, and highly effective in small doses.

### BACKGROUND OF THE INVENTION

Several natural phytonutrients, bioactives and antioxidants are being used in the nutraceutical and pharmaceutical industries for their health benefits. For example polyphenols from grape seed, pine bark and garlic are commonly used in nutraceutical formulations. There are several potent bioactives and micronutrients present in rice bran and rice germ oil. The unsaponifiable fraction (non-fat portion) of rice bran oil has the highest concentration (compared to other vegetable oils) of micronutrients and antioxidants such as tocopherols, tocotrienols, gamma oryzanol, phytosterols, polyphenols and squalene. Several clinical studies with rice bran oil demonstrated significant hypocholesterolemic, hypolipidemic and anti-atherogenic properties (Table-1). These beneficial properties are attributed to the potent micronutrients and antioxidants present in the unsaponifiable fraction of the oil (Table-2). The individual constituents of the rice bran oil (RBO) unsaponifiable fraction have been well studied for their hypolipidemic, hypoglycemic, hypocholesterolemic, antioxidant and other health benefits in animals and in human subjects.

TABLE 1
Selected RBO clinical studies
Clinical Study Summary
Effect of RBO Micronutrients on Serum Cholesterol

| | | READING | | |
|---|---|---|---|---|
| METHODOLOGY | PARAMETERS | (mg/dl) | CHANGE (%) | REFERENCE |
| Rice Bran Oil at 60 g/day. | Total Cholesterol | 219 | (19) | Lichtenstein, A. H., Ausman, L. M., Carrasco, W., |
| 15 moderately hypercholesterolomic | LDL-C | 162-122 | (25) | Gualtieri, L. J., Jenner, J. L., Ordovas, J. M., Nicolosi, |
| (mean 237 mg/dl) subjects. | HDL-C | no change | no change | R. J., Goldin, B. R., Shaefer, E. J. (1994) “Rice bran |
| 32 days, double blind crossover latin | Triglycerides | 131-109 | (17) | oil consumption and plasma lipid levels in |
| square design along with canola, corn | | | | moderately hyper cholesterolemic humans.” |
| and olive oils. | | | | Arteriosclerosis & Thrombosis Vol 14: 549556. |
| Rice Bran Oil at 35 g/day. | Total Cholesterol | 247-183 | (25) | Raghuram, T., Brahmaji Rao, U., Rukmini, C. |
| 12 hyper-cholesterolemic subjects | Triglycerides | 349-212 | (35) | (1989) “Studies on hypolipidemic effects of dietary |
| 30 days, with a control group of 9 | | | | Rice Bran Oils in humans.” Nutrition Reports |
| hyper-cholesterolemic subjects with | | | | International. Vol 39(5): 889-895. |
| peanut oil | | | | |
| Rice Bran Oil at 60 g/day. | | | | Suzuki, S., &Oshima, S. (1970) Influence of |
| 50 healthy females of normal | | | | blending of edible fat and oils on serum cholesterol |
| cholesterol. | | | | levels. Japanese J. NutritionVol 28(1): Part 1 pp3-6 |
| | | | | Part 2 pp194-198. |
| 7 days without blend. | Total Cholesterol | 194-164 | (15) | |
| 7 day blend with Safflower Oil (SO) | | | | |
| At RBO:SO of 70:30. | Total Cholesterol | 164-121 | (26) | |
| At RBO:SO of 85:15. | Total Cholesterol | 194-164 | (19) | |

TABLE 2
Selected study on RBO unsaponiflable fraction
EFFECT OF UNSAPONIFIABLE FRACTION ON SERUM CHOLESTEROL

| | | Readings | | | |
|---|---|---|---|---|---|
| | Lipid | Initial | Final USF/ | Change % | |
| Methodology | Parameters | (mg/dl) | RBO (mg/dl) | USF/RBO | References |
| 0.4% RBO Un-Saponifiable Fraction | Total | 374 | 243/288 | (35)/(23) | Sharma S D., Rukmini C.; |
| (USF) vs. equivalent 10% Rice Bran | Cholesterol | | | | Indian J Med Res. 1987 |
| Oil (RBO) studied in rats for | LDL + VLDL | 331 | 195/240 | (41)/(27.5) | Mar; 85: 278-81. |
| hypocholesterolemic effect. | HDL | 43 | 48/48 | 11.6/11.6 | |

Antioxidant defense mechanisms in a biological system play a major role in the prevention of a number of diseases, including cardiovascular, cerebrovascular, carcinogenic, and other metabolic age related disorders. Oxygen is essential to sustain life, but the reactive oxygen species known as singlet oxygen or free radical is a negatively charged electron state, which is dangerous to life. These free radicals are highly active, attack all the cellular components and can cause damage to DNA, alter the normal metabolism resulting in disease state. We are under constant challenge by these free radicals, unless we are charged with sufficient antioxidants to quench these free radicals, a slow damage to the body can result in disease state. Antioxidants provide a strong defense mechanism and help in preventing and arresting the progression of diseases. It is a constant battle to maintain the delicate balance between oxidant and antioxidants in the body. Epidemiological evidences are mounting on the significant role of natural antioxidants and their vital role in maintaining health and preventing diseases.

The constituents of the unsaponifiable fraction of RBO as a whole in their natural form act in synergy to produce a far more potent and greater hypolipidemic and hypocholesterolemic effect than when isolated into individual components. This synergistic mode of action of the RBO unsaponifiable constituent is shown in FIG.-1. Taking the lead from prior studies on RBO unsaponifiable constituents, the present inventors have developed an economically viable technology to obtain the micronutrients and antioxidants in the unsaponifiable fraction of rice bran and or rice germ oil in several fold concentration from the by-products of the said oil refining industry. These natural micronutrients and antioxidants are captured, extracted and concentrated without disrupting the natural lipid matrix within which they exist. Since these micronutrients and antioxidants are in their natural lipid matrix, they are far more bioavailable and potent than in their purified isolated form. These bioactive micronutrients and antioxidants as obtained and concentrated from the unsaponifiable fraction of rice bran oil or rice germ oil in their natural matrix is termed as Ri-Active™. The current invention specifically pertains to Ri-Active™ (as derived from rice bran oil or rice germ oil) but also generally extends to other vegetable oils such as palm oil, safflower oil, sunflower oil, peanut oil, soybean oil, coconut oil, rapeseed oil and other vegetable oils.

Various scientists have researched the hypocholesterolemic, hypolipidemic and other health beneficial properties of the unsaponifiable fraction of rice bran oil as a whole. Table-3 below shows results of one such human study performed using the RBO unsaponifiable fraction. The current inventors have taken the lead from such prior research to develop Ri-Active™ which is a far greater concentrate of that used in the Watkins study shown in Table-3 and at a far less expensive price. Ri-Active™ is a proprietary novel product for the prevention and treatment of cardiovascular diseases and other health disorders. The current invention pertains to the novel use of Ri-Active™, (and other similar extracts of vegetable oils mentioned above) derived from the by-products of the RBO (and other vegetable oil) industries, in the treatment and prevention of cardiovascular diseases and other health disorders.

TABLE 3
Effect of RBO non-saponifiable fraction in hypercholesterolemic subjects
Effect of RBO unsaponifiable fraction on hypercholesterolemic humans

| Methodology | Lipid Parameters | Reduction/Increase | | Reference |
|---|---|---|---|---|
| 50 hypercholesterolemic | Total Cholesterol | −14.1% | (p < 0.05) | T. R. Watkins, M. Geller, |
| subjects received in random | LDL-Cholesterol | −20.6% | (p < 0.05) | D. K. Kooyenga and M. L. |
| blinded fashion 3.1 g of | HDL-C/Total | +41.17% | (P < 0.025) | Bierenbaum; Environmental |
| RBO non-saponifiables or | Cholesterol | −43.98% | (P < 0.05) | and Nutritional Interactions, |
| placebo for 12 months. | Triglycerides/HDL-C | | | 3: 115-122, 1999. |

Rice bran oil is obtained from rice bran, i.e. the mesocarp of paddy. It is different from other vegetable oils as other vegetable oils are obtained from the seed/nuts. Palm oil, coconut oil, Olive oil and rice bran oil are obtained from the mesocarp of the fruit. These oils are rich in several natural antioxidants. The processing of these oils to an edible grade is complicated and the valuable bioactive micronutrients and antioxidants get destroyed during normal refining steps. Therefore, obtaining a micronutrient and antioxidant rich rice bran oil unsaponifiable fraction has posed various challenges to the oil technologist.

Japan, Korea, China, Indonesia, Thailand and India are the major producers of rice bran oil in the world. During the processing of rice bran oil many of the valuable micronutrients and antioxidants get striped off resulting in an oil of little nutritional value. Edible oil refining processes yield several by-products such as soap stock, distillate and gums. These by-products contain high concentrations of several of the vegetable oils unsaponifiable constituents. Although there are several technologies available for isolation of individual constituents and actives in the unsaponifiable fractions of rice bran oil (and other vegetable oils) such as tocopherols, tocotrienols and gamma Oryzanol etc., there are no references available in the literature on the therapeutic applications of the whole food extract concentrate containing the micronutrients in the unsaponifiable fraction of the said vegetable oils, in their natural form by itself. U.S. Pat. No. 5,660,691 discloses a technology to isolate the valuable tocopherols and tocotrienols from the rice distillate. U.S. Pat. No. 5,288,902 discloses a technology to isolate Gamma Oryzanol, another valuable antioxidant from the soap stock, which is thrown away as a waste product. Isolating the micronutrient or antioxidants disrupts the natural matrix and the potency and bioavailability is lost. So far nobody has ever attempted to isolate, concentrate and exploit the bioactives from the unsaponifiable fraction of rice bran oil or rice germ oil in their natural matrix as a whole as derived from the by-products of oil refining or bran processing. The current invention demonstrates the therapeutic usefulness of the whole unsaponifiable micronutrient and antioxidant extract concentrated from rice bran oil by-products and/or rice germ (rice bran by-product) with out destroying the natural matrix to isolate individual constituents. Ri-Active™ is a whole food extract concentrate of the unsaponifiable fraction of rice bran and/or rice germ oil containing all the micronutrients and antioxidants from rice bran and/or rice germ in a concentrated form.

### BRIEF SUMMARY OF THE INVENTION

Cardiovascular disease is a leading cause of mortality in the United States and in developed countries around the world. According to the Center for Disease Control and Prevention, this condition affects over 61 million Americans and accounts for nearly 40% of all deaths. The treatment cost of CVD in the United States exceeds $300 billion annually. According to the American Heart Association 1 in 5 Americans have some form of CVD. The disease is linked with well-defined risk factors, such as lipid anomalies, arterial hypertension, diabetes, obesity and smoking. The estimated breakdown of the CVD patient population is as follows:
High blood pressure: 50.0 million
Coronary heart disease: 13.9 million
Congestive heart failure: 4.7 million
Stroke: 4.0 million
Rheumatic heart disease: 1.8 million

There is an escalating need to contain this disease effectively without exponentially increasing the associated healthcare costs. Prescription medications alone will not suffice in addressing this need as they have a high direct and indirect cost associated with them. Alternative strategies to fight these conditions and their related health problems have to be considered. Beyond cost, there are also significant side effects associated with prescription drugs and medical practitioners are reluctant to increase dosages of these drugs unless absolutely needed. Statin Drugs for lowering cholesterol have several side effects.

The current invention (a novel solution): The current inventors aim to address this problem by providing the much-needed safe and effective alternative for lowering cholesterol, controlling blood pressure and preventing/treating other health disorders. The current inventors intend to formulate powerful supplements using their proprietary technology to concentrate micronutrients from by-products of the rice bran oil processing industry (and other vegetable oil refining industries) and use this concentrate in a novel way for the prevention and treatment of diseases. The current inventors have developed a proprietary technology to innovatively use the by-products of vegetable oil refining industry in the treatment and prevention of diseases. This invention has been validated by conducting an animal study (described below) in moderately hypercholesterolemic hamsters.

### BRIEF DESCRIPTION OF THE FIGURES

FIG. 1:

This figure shows the various steps in the cholesterol homeostasis pathway, also known as the HMGCoA reductase pathway. This figure highlights the modes of action of each of the key unsaponifiable constituents of rice bran oil (or rice germ oil) on the cholesterol homeostasis pathway. This synergistic mixed mode of action is responsible for the highly effective hypocholesterolemic and hypolipidemic action of Ri-Active™ at very low concentrations. This synergy is demonstrated in the study conducted by the current inventors.

FIG. 2:

This figure shows the performance of Ri-Active™ as compared to a placebo high cholesterol diet. The comparison is based on a measure of total cholesterol reduction in hamsters after 9 weeks of administering the treatment.

FIG. 3:

This figure shows the performance of Ri-Active™ as compared to a placebo high cholesterol diet. The comparison is based on a measure of LDL—cholesterol reduction in hamsters after 9 weeks of administering the treatment.

FIG. 4:

This figure shows the performance of Ri-Active™ as compared to a placebo high cholesterol diet. The comparison is based on a measure of HDL—cholesterol in mg/dL in hamsters after 9 weeks of administering the treatment.

FIG. 5:

This figure shows the performance of Ri- Active™ as compared to a placebo high cholesterol diet. The comparison is based on a measure of blood glucose levels in hamsters after 9 weeks of administering the treatment.

### DETAILED DESCRIPTION OF AN EMBODIMENT OF THE PRESENT INVENTION

Although this invention will be described by way of example and with reference to preferred embodiments thereof, it is to be understood that modifications or improvements may be made thereto without departing from the scope or spirit of the invention. It should be noted that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be included within the present invention.

The by-products of the RBO and other Vegetable oil refining industries typically contain high concentrations of the unsaponifiable fraction constituents of the vegetable oil. These rice bran oil (and other vegetable oil) refining industry by-products are one source for economically obtaining and concentrating the unsaponifiable fraction of the oil. Alternately rice germ obtained as a by-product of the rice bran milling industry is another economical source for obtaining and concentrating the unsaponifiable fraction constituents. The current inventors have developed a proprietary technology for extracting and concentrating Ri-Active™ from the above by-products of the RBO or rice germ industries. The by-products of RBO (and other vegetable oil) refining industry are typically discarded or sold to the soap industry for their free fatty acid contents. The nutraceutical and functional food industry also uses such oil refining industry by-products to isolate and purify individual phytonutrients of value which are then used in nutraceutical formulations. These techniques are expensive and yield purified and isolated micronutrients which have diminished bioavailability.

The current inventors have developed a commercially viable and inexpensive technology to concentrate the therapeutically useful unsaponifiable micronutrient rich fraction from the by-products of rice bran oil refining with out destroying the natural matrix within which the key bioactive molecules and antioxidants exist in their natural form. The resulting product known as Ri-Active™ is a very valuable concentrate of several of the unsaponifiable micronutrients and antioxidants of rice bran oil. Alternatively the current inventors can also prepare Ri-Active™ from rice germ while retaining the unsaponifiable micronutrients and antioxidants in a highly bioavailable form without disruption to natural matrix. Ri-Active™ is a novel micronutrient and antioxidant concentrate which can be stabilized and directly used in several pharmaceutical, nutraceutical and health food formulations for treating and preventing diseases such as high blood pressure, diabetes, obesity, cardiovascular disease, liver disorders, cancer of several organs, arthritis, deficient immune function and other disorders.

Ri-Active™ prepared by this method is more cost effective (than blending similar concentrations of individually procured antioxidants and micronutrients) and safe. Ri-Active™ prepared in this method is far more efficacious and bioavailable than blending isolated constituents in similar concentrations. Ri-Active™ when stabilized can be used as the basic ingredient for the pharmaceutical, nutraceutical and health food formulations because blending the individually prepared antioxidants and other micronutrients in the required quantities is highly uneconomical and ineffective due to diminished bioactivity of the isolated entities. The bioavailability of these micronutrients and antioxidants in their natural form as present in the rice bran oil, rice germ oil and other vegetable oils is superior to that of individually isolated components as shown in the study conducted by the current inventors.

The current inventors have developed a technology to produce Ri-Active™ at an estimated cost of $0.02 to $0.03 per serving (to bring about the health benefits such as cardiovascular disease risk factor reduction etc. described below). This is cost is several times lower than pharmaceutical compounds which produce similar health benefits. Further, the cost of delivering a mixture of procured constituents of Ri-Active™ is many times greater and far less effective in lowering cholesterol and providing the below mentioned health benefits. As an example, tocotrienols retail for around $200 per Kg and gamma oryzanol retails for around $75 per Kg. Other constituents are similarly expensive and therefore the cost of mixing procured constituents for delivering an equivalent dosage as that contained in Ri-Active™ is prohibitively expensive.

As an extension, the inventors can implement the same proprietary technology used to produce Ri-Active™ from RBO refining by-products or rice germ to produce similar health beneficial unsaponifiable fraction concentrates from the by-products of other vegetable oil refining industries. The vegetable oils referenced herewith are rice bran, palm, safflower, sunflower, corn, coconut, soybean, rape seed, pea nut and cotton seed oils and the germ oils of the said vegetable oils. By example of the studies performed by the current inventors on Ri-Active™ to show its enhanced health benefits, this patent extends to the use of similar novel concentrates derived from other vegetable oil refining industry by-products in the treatment and prevention of health disorders.

Ri-Active™ has many of the valuable micronutrients and antioxidants contained in the RBO unsaponifiable fraction. Table-4 below quantifies the key therapeutic micronutrients contained in Ri-Active™ as a whole concentrate of the unsaponifiable fraction (not a mixture of isolated purified components) within the natural matrix of RBO (or rice germ oil).

TABLE 4
Composition of Ri-Active ™

| Unsaponifiable constituent | % by weight |
|---|---|
| Tocopherols (α, β, γ, δ isomers) | 1-15% |
| Tocotrienols (α, β, γ, δ isomers) | 1-15% |
| Phytosterols (β-sitosterol, sitostenol, campesterol, | 1-40% |
| stigmasterol and 27 other sterol derivatives) | |
| Polyphenols (trans ferulic acid, protocatechuic acid, | 0-20% |
| epicatechin, p-coumaric acid, sinapic acid, tricin, | |
| a flavones, GABA and 8 other polyphenols) | |
| Squalene | 0-10% |
| Gamma aminobutyric acid (GABA) | 0-10% |
| Gamma (γ) oryzanol | 0-40% |
| Polycosanol | 0-10% |
| Total unsaponifiable content | 10-80% |

Summary of Therapeutic Benefits:

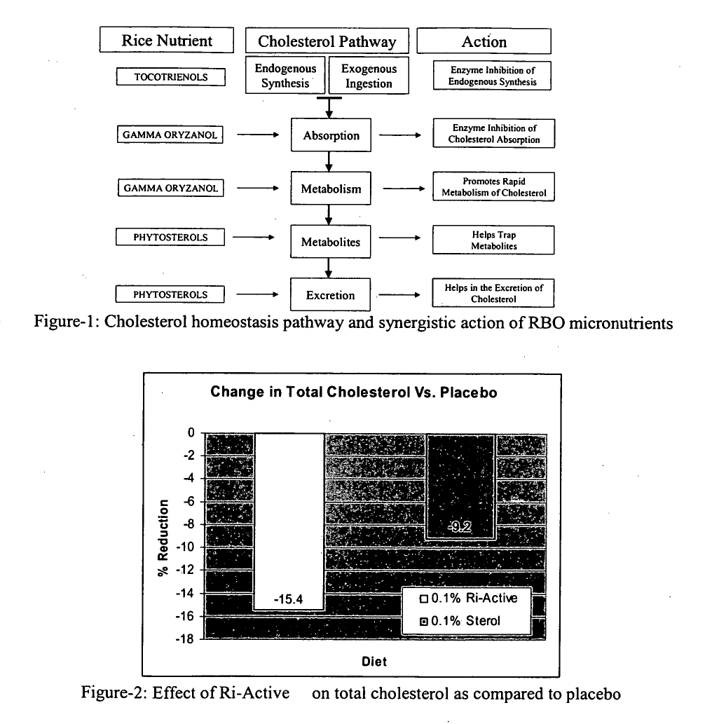

Rice bran oil and rice germ oil have a high unsaponifiable fraction and have been evaluated for their nutritional and therapeutic value by several groups of scientists from Japan, India and USA. Rice Bran Oil demonstrated significant hypocholesterolemic effect (Table-1) in human subjects with high cholesterol. There are several clinical studies on the lipid lowering effect of RBO and cardiovascular disease risk parameters in hypercholesterolemic individuals. The unsaponifiable fraction of rice bran oil alone is responsible for the significant hypocholesterolemic effect (Table-2), indicating that the combined antioxidants present in the unsaponifiable fraction of rice bran oil are responsible for this effect. The synergistic mechanism by which these antioxidants work in bringing down the circulating cholesterol and other lipid parameters are also scientifically studied and proven (FIG. 1).

Tocotrienols exert hypocholesterolemic action in humans and animals. Lovastatin, a hypolipidemic pharmaceutical drug and tocotrienols work by suppressing the activity of β-hydroxy-β-methylglutaryl coenzyme-A reductase (HMGCoA) through different mechanisms (post transcriptional vs. Competitive inhibition). A human study with 28 hypercholesterolemic subjects in 5 phases of 35 days each with tocotrienol rich fraction from RBO alone and in combination with lovastatin has been conducted (Qureshi et al., 2001). In this study tocotrienol/lovastatin alone effectively reduce serum total cholesterol by (14%, 13%) respectively and LDL-C by (18%, 15%) respectively, while the combination of 10 mg of lovastatin with 50 mg of tocotrienol fraction from rice bran oil resulted in significant improvement of lipid parameters 20% and 25% (p<0.001) in these subjects. Significant improvement in HDL/LDL ratio was also demonstrated in these subjects.

The above findings and prior research conducted (Table-3) on the constituents of the unsaponifiable fraction of RBO led the current inventors to believe that the concentrated extract of this unsaponifiable fraction of vegetable oils (especially RBO and rice germ oil) which is rich in bioactives, micronutrients and antioxidants would be cost effective and highly efficacious in the treatment of cardiovascular and other diseases. Therefore, the current inventors developed a cost effective and commercially viable process to extract and concentrate this therapeutic unsaponifiable fraction from RBO and/or rice germ oil (as well as other vegetable oils) by means of a mixed chemical/non-chemical process in a novel way. The current invention covers the uses of Ri-Active™ (and other whole extract concentrates of vegetable oil unsaponifiable fractions) in the treatment and prevention of diseases as demonstrated by the studies conducted by the inventors.

Results of Discovery:

The present applicants were able to successfully isolate Ri-Active™, the concentrated unsaponifiable portion of rice bran oil in its natural matrix. The Analysis of Ri-Active™ showed excellent potential for use in pharmaceutical and nutraceuticals formulations. Ri-Active™ is nothing but the unsaponifiable fraction of rice bran and/or rice germ oil in a highly concentrated form and was obtained by implementing a novel and commercially viable process developed by the current inventors using a sourced by-product from the RBO industry. The current inventors are also able to obtain Ri-Active™ from rice germ and crude rice bran oil in a commercially viable way.

As an example for a technique to obtain Ri-Active™, the current inventors sourced a qualified sample of deodorizer distillate (a by-product of rice bran oil refining). The free fatty acid contained in this sample of deodorizer distillate was converted into its lower alkyl ester using conventional methods. This free fatty acid can also be neutralized and removed by a mild alkali treatment. The resulting product was subjected to short path distillation to remove the alkyl ester of the free fatty acid. The residual product (Ri-Active™), was purified and stabilized in a lipid medium and it contained the concentrated unsaponifiable bioactives, micronutrients and antioxidants present in rice bran oil. Table-5 below shows a summary of the concentrations of the key components of this sample preparation of Ri-Active™ which the current inventors produced and tested in the below mentioned animal study.

TABLE 5
Contents/Composition of Ri-Active ™

| Ri-Active ™ Components | g/100 g |
|---|---|
| Tocopherols (α, β, γ, δ isomers) | 4 g/100 g |
| Tocotrienols (α, β, γ, δ isomers) | 7 g/100 g |
| Phytosterols (β-sitosterol, sitostenol, campesterol, | 7 g/100 g |
| stigmasterol and 27 other sterol derivatives) | |
| Polyphenols (trans ferulic acid, protocatechuic acid, | 1 g/100 g |
| epicatechin, p-coumaric acid, sinapic acid, tricin, a | |
| flavonol, GABA and 8 other polyphenols) | |
| Gamma oryzanol | 30 g/100 g |
| Squalene and other hydrocarbons | Trace amounts |

Hypolipidemic, Hypocholesterolemic and Hypoglycemic effect of Ri-Active™ (A Study in Syrian Hamsters): A pilot study was conducted by the current inventors in hamsters to establish the efficacy and safety of Ri-Active™. Rice bran oil unsaponifiable fraction was obtained for the first time in its natural matrix and in several fold higher concentration than in rice bran oil by a proprietary technology from the by-products of the RBO refining industry. This product (Ri-Active™) contained all the bioactive molecules of rice bran oil, such as tocopherols (4%), tocotrienols (7%), gamma oryzanol (30%), phytosterols, phytostanols (1%), polyphenols of hydroxyl-cinnamic acid derivatives, squalene, gamma amino butyric acid all embedded in the natural lipid matrix of rice bran oil and is named as Ri-Active™.

A pilot study was undertaken to evaluate the efficacy and safety of Ri-Active™. Syrian golden hamsters (60) were fed semi-purified diets containing coconut oil and 0.5% cholesterol to induce moderate hypercholesterolemia and divided into 6 groups of 10 animals each. Group 1 (Placebo) continued to be fed the same control diet. Group 2 (Positive control) was fed 0.1% phytosterols in the control diet (FDA approved natural hypocholesterolemic agent) Group 3 was fed 0.5% phytosterols in the control diet, Group 4 was fed 0.1% Ri-Active™ in the control diet, Group 5 was fed 0.5% Ri-Active™ in the control diet, and Group 6 was fed 1% Ri-Active™ in the control diet. All diets were prepared by the Research diets, Pennsylvania USA. All animals were fed the corresponding diets for 9 weeks. Weekly body weights were monitored. Physical symptoms, if any and gastrointestinal function were also monitored in all the animals. Blood was drawn by retro orbital sinus at the end of 4, 6 and 9 weeks, from each animal and analyzed for total cholesterol, LDL-C, Triglycerides, HDL-C, serum glucose levels AST and ALT.

The Watkins clinical study (Table-3) conducted on 50 human subjects over a 12 month period showed that administering 3.1 g/day of RBO unsaponifiables resulted in a 14.1% reduction in total cholesterol and a 20.5% reduction in LDL-C in addition to an increase in HDL-C levels and significant decrease in triglycerides levels. The inventors hypothesized that Ri-Active™ with ten times the concentration of that used in Watkin's study, having multiple bioactives from the RBO unsaponifiable fraction would act synergistically resulting in the overall cardiovascular risk benefits and would lower LDL cholesterol to an even greater extent while increasing the HDL-C levels.

Plant phytosterols are currently the only natural products in the market with a FDA health claim for cardiovascular health (at least 800 mg of phytosterols or 1300 mg of phytosterol esters in two meals per day). Phytosterols compete with cholesterol uptake releasing cholesterol for excretion. The average decline in LDL-c levels through the use of phytosterols is thought to be between 8%-15% in humans.

Test Results: (Comparison Between Groups):

The results indicated significant hypocholesterolemic, hypolipidemic and hypoglycemic effect of Ri-Active™ compared to phytosterols (which is the positive control or “gold standard”) as well as the placebo control (no treatment) group.

0.1% Ri-Active™ (˜10 mg dosage): In the 0.1% Ri-Active™ group, total cholesterol levels were 16% lower (242 mg/dl) and statistically significant (P<0.008) when compared to placebo group (287 mg/dl) and more than 6% greater reduction than the 0.1% phytosterols group (260 mg/dl). The significance of this result is further underscored because the 0.1% Ri-Active™ group contains less than 1/10^{th} the concentration of phytosterols as compared to the 0.1% phytosterol group. FIG. 2 shows a comparative performance of Ri-Active™ as measured by total cholesterol reduction.

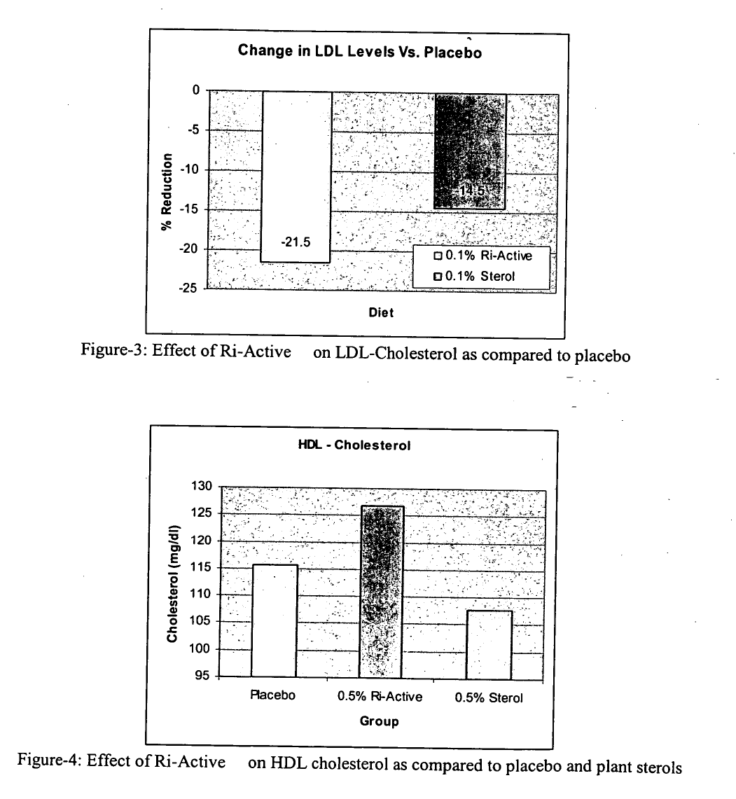

The decrease in LDL-C in this group (135 mg/dl) was remarkable and significant (P<0.001) with 22% reduction when compared to placebo group (171 mg/dl) and nearly 8% greater reduction when compared with 0.1% phytosterol group (146 mg/dl) as seen in FIG. 3.

Triglycerides also showed an excellent reduction of 21% (242 mg/dl) when compared with placebo (306 mg/dl) and this was 12% greater to reduction shown by the 0.1% phytosterol group (279 mg/dl).

0.5% Ri-Active™ (˜50 mg dosage): Increasing Ri-Active™ dosage by 5 times showed a significant increase in HDL-C levels. HDL-C levels in this group (127 mg/dl) increased by 10% more than the placebo group (115 mg/dl) and 17.6% more than the 0.5% phytosterol group (108 mg/dl). These results were statistically significant with P<0.022 (FIG. 4). This is the first natural product to demonstrate an increase in HDL-C along with a significant decrease in LDL-C. This finding is strongly supported by previously published human clinical studies conducted over a 12-month period with RBO unsaponifiable fraction (Watkins et al. in 1990: Table-3). The phytosterol groups showed a reduction in HDL-C levels as predicted by previous studies.

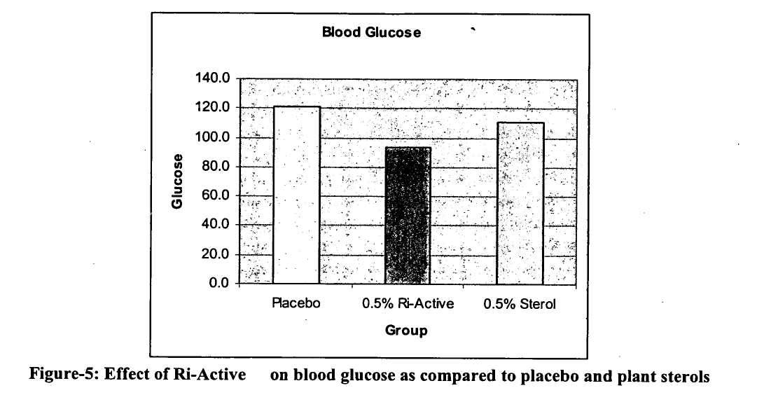

Increasing Ri-Active™ dosage by 5 times also showed significant hypoglycemic effect (P<0.015) by lowering blood glucose levels to 93 mg/dl which was 23% lower compared to the placebo diet (121 mg/dl) and 15.6% reduction compared to the 0.5% phytosterol treatment group (111 mg/dl). These results were statistically significant with P<0.030 (See FIG. 5).

Increasing the phytosterol dosage by 5 times (0.5% phytosterol group) did not show any statistically significant reduction in either total cholesterol or LDL-C as compared to the 0.1% Ri-Active™ group (0.1% Ri-Active™ group had 1/50^{th} the concentration of phytosterols as compared to the 0.5% phytosterol group). Further, there was no statistical significance in the total cholesterol, LDL-C and Triglycerides reduction between the 0.5% phytosterol group and 0.5% Ri-Active™ groups.

Animals as their own Control: The group comparisons above indicate a statistically significant improvement in lipid parameters for the 0.1% Ri-Active™ group, the 0.5% Ri-Active™ group and the 0.5% phytosterol group (0.5% phytosterol group is superior to 0.1% phytosterol group as predicted by prior studies).

TABLE 6
Lipid parameters of Placebo group

| Parameter | Peak (Week 4) | Final (Week 9) | Change (%) |
|---|---|---|---|
| Total Cholesterol | 396 +/− 87 | 287 +/− 26 | −27.5 |
| LDL-C | 272 +/− 77 | 171 +/− 18 | −37.1 |
| HDL-C | 124 +/− 30 | 115 +/− 15 | −7.2 |
| Triglycerides | 470 +/− 220 | 306 +/− 111 | −34.9 |
| Glucose | 125 +/− 28 | 121 +/− 15 | −3.2 |

TABLE 7
Lipid parameters of 0.1% Ri-Active

| Parameter | Peak (Week 4) | Final (Week 9) | Change (%) |
|---|---|---|---|
| Total Cholesterol | 381 +/− 141 | 243 +/− 36 | −36.2 |
| LDL-C | 271 +/− 166 | 135 +/− 21 | −50.1 |
| HDL-C | 110 +/− 20 | 108 +/− 20 | N.C |
| Triglycerides | 379 +/− 286 | 242 +/− 69 | −36.1 |
| Glucose | 126 +/− 67 | 110 +/− 17 | −12.8 |

TABLE 8
Lipid parameters of 0.5% Ri-Active

| Parameter | Peak (Week 4) | Final (Week 9) | Change (%) |
|---|---|---|---|
| Total Cholesterol | 436 +/− 126 | 279 +/− 61 | −36 |
| LDL-C | 322 +/− 109 | 153 +/− 41 | −52.4 |
| HDL-C | 115 +/− 24 | 127 +/− 21 | 10.3 |
| Triglycerides | 532 +/− 224 | 291 +/− 148 | −54.7 |
| Glucose | 121 +/− 42 | 93 +/− 8 | −22.7 |

TABLE 9
Lipid parameters of 0.5% Phytosterols group

| Parameter | Peak (Week 4) | Final (Week 9) | Change (%) |
|---|---|---|---|
| Total Cholesterol | 340 +/− 143 | 279 +/− 26 | −18 |
| LDL-C | 233 +/− 134 | 134 +/− 18 | −42.5 |
| HDL-C | 108 +/− 16 | 108 +/− 11 | N.C |
| Triglycerides | 440 +/− 346 | 259 +/− 126 | −41.1 |
| Glucose | 122 +/− 47 | 111 +/− 21 | −9.5 |

TABLE 10
Ratio Comparison of Treatment Groups

| | |
|---|---|
| TC/HDL-C | LDL-C/HDL-C |

| Treatment | Initial | Final | Initial | Final |
|---|---|---|---|---|
| Placebo | 3.28 | 2.51 | 2.28 | 1.51 |
| 0.1% Ri-Active | 3.40 | 2.25 | 2.45 | 1.25 |
| 0.5% Ri-Active | 3.78 | 2.19 | 2.79 | 1.20 |
| 0.5% Phytosterols | 3.15 | 2.58 | 2.16 | 1.24 |

Safety:

Ri-Active™ showed good animal growth performance and health maintenance at very high doses. It did not show any adverse effects or any physical signs and symptoms of toxicity. Further, the AST and ALT measurement made by increasing the Ri-Active™ dosage by 10 times (1% Ri-Active™) were not different from the control group and indicated its safety.

Observations:

Ri-Active™ at 0.1% (10 mg Ri-Active™ in the diet) contains 1.1 mg Tocos (Tocopherol and Tocotrienols), 3.0 mg Gamma Oryzanol and less than 1.0 mg of phytosterols. In the light of these low bioactive concentrations, the observed significant hypolipidemic effect in 0.1% Ri-Active™ group is truly remarkable. It shows the synergistic effect of RBO bioactives acting in concert at very low concentration in their natural matrix. Previous studies on individually purified RBO bioactives have never shown any hypocholesterolemic or hypolipidemic at such low concentrations.
1. Ri-Active™ at 5 times the concentration (0.5%) demonstrated a significant increase in HDL-C, which is a positive factor to reduce the risk of CVD. This is the first reported natural product to increase HDL-C.
2. Ri-Active™ at 5 times the concentration (0.5%) demonstrated a significant hypoglycemic effect, which is again risk factor of cardiovascular disease. This is an incidental but highly positive finding in this study.
3. In our current study we have clearly demonstrated that Ri-Active™ is safe and that it is superior to plant phytosterols (the gold standard) for the reduction of CVD risk factors. We have since located and tested a better source of RBO by-product for manufacturing Ri-Active™ and are confident that our future results in human subjects would show further improvement.

These results indicate significant hypocholesterolemic, hypolipidemic and hypoglycemic effect of 0.1% Ri-Active™ compared with 0.1% phytosterols which is a “gold” standard, as well as with placebos. Total cholesterol levels were lower and statistically significant (P value between groups is 0.010). It has shown a reduction of 16% compared to placebo; and more than 6% reduction compared to phytosterols @ 1/10th the sterol concentration, which is statistically significant (P value between groups less than 0.001). The decrease in LDL-C was 22% when compared with placebo; and nearly 8% decrease when compared with 0.1% phytosterols @ 1/10th the sterol concentration. Increasing Ri-Active™ dosage by 5 times (0.5% Ri-Active™ group) showed a significant increase of HDL-C levels by 10% over the placebo (high cholesterol diet) group. This is the first natural product to demonstrate an increase in HDL-C, which is a well-recognized positive factor in controlling CVD. Increasing Ri-Activeυ dosage by 5 times (0.5% Ri-Active™ group) also showed significant hypoglycemic effects by lowering blood glucose levels by 23% over the placebo group. Ri-Active™ showed good growth performance and did not show any adverse effects by any signs and symptoms or by the AST and ALT measurement which were not different from the control group indicating its safety.

Conclusion:

The results of the pilot experiment concludes that
1. 0.1% Ri-Active™ (10 mg of Ri-Active™ in the diet) includes 1.1 mg Tocos, 3.0 mg Gamma Oryzanol and less than 1.0 mg of phytosterols. The observed results are remarkable and significant showing a synergistic effect of RBO bioactives acting in concert at very low concentration in their natural matrix.
2. Ri-Active™ at 5 times the concentration (of 0.1%,), demonstrate significant raise in HDL-C, which is a positive factor to reduce the risk of CVD. This is the first reported natural product to increase HDL-C.
3. Ri-Active™ at 5 times the concentration (of 0.1%) demonstrated significant hypoglycemic effect, which is again a factor to reduce the risk of cardiovascular disease. This is an incidental finding in this study.
4. Ri-Active™ at 5 times the concentration (of 0.1%), demonstrate significant raise in HDL-C, which is a positive factor to reduce the risk of CVD. This is the first reported natural product to increase HDL-C.
5. Ri-Active™ at 5 times the concentration (of 0.1%) demonstrated significant hypoglycemic effect, which is again a factor to reduce the risk of cardiovascular disease. This is an incidental finding in this study.
6. Ri-Active™ demonstrated to be safe without any adverse effects and more efficacious than the “gold” standard, phytosterol.

The inventors contend that the above tested Ri-Active™ can be used in the various pharmaceutical, nutraceutical, cosmeceutical and health food dietary supplements for preventing and treating several health disorders such as high blood pressure, hypercholesterolemia, hyperlipidemia, cardiovascular disease, cerebrovascular disease, diabetes, cancer, obesity, inflammatory diseases, arthritis, improve immune function, used as a sports and weight loss formulations in improving lean body mass and liver disorders. Ri-Active™ can be used in skincare, hair growth, UV protective, antidandruff properties and cosmaceutical properties. Ri-Active™ has a very wide range of application as mentioned above.

### REFERENCES

All references cited in this application are incorporated herein by reference.

1. Nicolosi R. J., Ausman L. M., and Hegstead M. (1991) “Rice bran oil lowers serum and LDL lipoprotein cholesterol and apo-B levels in non-human primates” Atherosclerosis. 88(2-3) 133-142.
2. Rukmini C., and Raghuram T. C., (1991) “Nutritional and biochemical aspects of the hypolipidemic action of rice bran oil: A review” J. Amer. Coll. Nutrition. 10: 366-375.
3. Sugano M., and Tsuji E., (1997) “Rice bran oil and cholesterol metabolism”. J. of. Nutrition, 127(3): 521S-524S
4. Qureshi A. A., Qureshi N., Hasler-Rapacz J. O., (1991) “Dietary tocotrienols reduce concentrations of plasma cholesterol, apolipoprotein B, thromboxane B2 and platelet factor 4 in pigs with inherited hyperlipidemias.” Am. J Clin. Nutr. 53:1021 S
5. Qureshi A. A., Bradlow B. A., Salser W. A., Brace L. D., (1997), “Novel tocotrienols of rice bran modulate cardiovascular disease risk parameters of hypercholesterolemic humans,” J. Nutr. Biochem 8: 1-9
6. Nesaratnam K., Stephen R., Dils R., Darbre P., (1998) “Tocotrienols inhibit the growth of breast cancer cells irrespective of estrogen receptor status.” Lipids, 33(5): 461-469.
7. Tomeo A. C., Geller M., Watkins T. R., (1995) “Antioxidant effects of tocotrienols in patients with hyperlipidemia and carotid stress.” Lipids, 30:1179
8. Xu Z, Hua N., Godber J. S., (2001), “ Antioxidant activity of tocopherols, tocotrienols, and gamma oryzanol components from rice bran against cholesterol oxidation accelerated by 2,21-azobis(2-methylpropionamideine) dihydrochloride.” J. Agric.Food.Chem, 49(4): 2077-81.
9. Ando Y., Pharcos I., (1984) “Effect of gamma oryzanol as sunscreen agent”, Jpn. J. Cosmetic Sci. Soc, (1) 41.
10. Akihisa T., Yasukawa K., Yamaura M., Ukiya M., Kimura Y., Shimizu N., and Arai K., (2000) “Triterpene alcohol and sterol ferulates from rice bran and their anti-inflammatory effects”, J. Agri. Food. Chem, 48:2313-2319.
11. Ieiri T., Kase N., Hashigami Y., Nakumura T., Shimoda S., (1982) “Effect of gamma oryzanol on the hypothalamo-pituitary axis in the rat”, Nippon Naibunpi Gokkai Zasshi 58(10):1350-6
12. Bouic P. J. D., Etsbeth S., Liebenberg R. W., Albrecht C. F, Pegel G., Van Jaarsveld P. P., (1996) “Beta-sitosterol and beta-sitosterol glucoside stimulate human peripheral blood lymphocyte proliferation: Implication for their use as an immunomodulatory vitamin combination”, International . J. Immunopharmacology, 18(12): 693-700.
13. Ann Hudson, Dinh P. A., Kokubun T., Simmonds S. J., and Gescher A., (2000) “Characterization of potentially chemopreventive phenols in extracts of brown rice that inhibit the growth of human breast and colon cancer cells”. Cancer Epidemiology, Biomarkers & Prevention. 9: 1163-1170.
14. Sunita T. Manorama R., and Rukmini C. (1996) “Lipid profile on blended oils with rice bran oil—A study in human subjects”. Report to Dabur India Ltd. Asia Pacific Journal of Clinical Nutrition
15. Sunita T., Manorama R. and Rukmini, C. (1997) “Lipid profile of rats fed a blend of rice bran oil in combination of safflower/sunflower oil.” Plant Foods for Human Nutrition 51: 219-230.
16. Reddy Sastry C. V., Rukmini C., Ike Lynch, and McPeak D., (1999) “Process for obtaining micronutrient enriched rice bran oil.” U.S. Pat. No. 5,985,344.
17. Rukmini C., (2000) In “Phytochemicals as Bioactive Agents.” Bidlack, W. R., Omaye, S. T., Meskin, M. S., Topham, D. K. W (Ed), Chapter 13 “Bioactives in Rice Bran and Rice Bran Oil.” 213-239.
18. Rukmini C., Reddy Sastry C., McPeak P., and Lynch I. (2000) “Method for treating hypercholesterolemia, hyperlipidemia, and atherosclerosis.” U.S. Pat. No. 6,126,943.
19. Patricia McPeak, Rukmini C., Reddy Sastry Cherukuri (2001), “Supportive therapy for Diabetes, Hyperglycemia and Hypoglycemia” U.S. Pat. No. 6,303,586 B1
20. Rukmini C., Reddy Sastry C. V., McPeak P., and Lynch I. (2002) “Method for treating hypercholesterolemia, hyperlipidemia, and atherosclerosis.” U.S. Pat. No. 6,350,473 B1
21. Rukmini C. (2003) Phytochemical Products: rice bran; Chapter 17, Pg347-376; in “Phytochemical Functional Foods” (Ed) Ian Johnson and Gary Williamson (CRC) Woodhead Publishing Ltd. (Great Briton).
22. Rukmini C., Patricia McPeak, Reddy Sastry V. Cherukuri, Ike Lynch and Qureshi A. (2003) “Method for treating hypercholesterolemia, hyperlipidemia, and atherosclerosis.” U.S. Pat. No. 6,558,714 B2
23. Rukmini C., Patricia McPeak, Reddy Sastry Cherukuri, Ike Lynch and Qureshi A., (2004) “Method for treating hypercholesterolemia, hyperlipidemia, and atherosclerosis.” U.S. Pat. No. 6,733,799 B2.
24. Watkins T. R., Geller M., Kooenga D. K. and Bierenbaum M. L. (1999) “Hypocholesterolemic and antioxidant effect of rice bran oil non-saponifiables in hypercholesterolemic subjects” Environmental and Nutritional Interactions, 3(2) 115-22

1. The use of a novel composition (Ri-Active™), containing a concentrate of the bioactives, micronutrients and antioxidants present in the unsaponifiable fraction of rice bran oil or rice germ oil, in the prevention and treatment of health disorders.

2. In claim 1, wherein the said composition (Ri-Active™) is a novel, whole extract concentrate of the unsaponifiable fraction of rice bran or rice germ oil and is derived from the rice bran oil industry by-products or rice germ oil using the proprietary technology developed by the current inventors.

3. In claim 1, wherein the said composition (Ri-Active™) is comprised of a variety of the naturally occurring bioactives, micronutrients and antioxidants of rice bran oil or rice germ oil in the range of concentrations shown in Table-3.

4. In claim 1, wherein the said naturally occurring unsaponifiable bioactives, micronutrients and antioxidants are present in the natural lipid matrix of rice bran oil and the unsaponifiable fraction is concentrated as a whole and not prepared as a mixture of individually isolated constituents.

5. In claim 1, wherein the said health disorders refer to one or more among hypertension, hyperlipidemia, obesity, inflammatory disease, arthritis, hypercholesterolemia, cardiovascular disease, cerebrovascular disease, arteriosclerosis, diabetes mellitus, immune dysfunction and cancer in mammals.

6. In claim 5, wherein the said mammals refer to humans, equines and animals.

7. A natural unsaponifiable concentrate of bioactives, micronutrients and antioxidants (Ri-Active™) derived from directly purifying and concentrating the by-products of rice bran oil refining or rice germ, without mixing isolated constituents, either natural or synthetic, has so far not been used for the prevention or treatment of health disorders mentioned in claim 5.

8. A method for lowering total serum cholesterol, LDL-C, apolipoprotein B, triglycerides, improving HDL-C levels, inhibiting platelet aggregation and dissolving aortic streaks in mammals by administering the composition (Ri-Active™) from claim 1.

9. A method of administering Ri-Active™, having a total concentration of bioactives, micronutrients, and antioxidants from 10% to 80% is effective in preventing and treating the said health disorders in claim 5.

10. The use of formulations for functional foods, medical foods, nutraceutical supplements, pharmaceuticals and cosmeceuticals prepared from Ri-Active™ by blending with other natural herbs, nutraceuticals and pharmaceuticals, in treatment and prevention of health disorders in claim 5.

11. In claim 10, wherein the said functional food formulations can be delivered as one or more among butter substitutes, margarines, salad dressings, mayonnaise, beverages, cereals, oils, health bars and snacks.

12. In claim 10, wherein the said functional food formulations can contain concentrations of 0.1% to 15% of Ri-Active™.

13. In claim 10, wherein the said pharmaceutical and nutraceutical formulations can be delivered in the form of tablets, capsules, creams, liquid syrups and lotions.

14. In claim 10, wherein the said cosmeceutical preparations can be delivered as skin softening creams, body lotions, suntan, UV blocking creams, and anti-dandruff shampoos.

15. The use of similar concentrated compositions, prepared from other vegetable oil by-products, using the same proprietary technology used by the current inventors to produce Ri-Active™ from rice bran oil by-products, for the prevention and treatment of health disorders.

16. In claim 15, wherein the said vegetable oils are one among palm, safflower, sunflower, corn, coconut, soybean, rape seed, pea nut and cotton seed oils and the germ oils of the said vegetable oils.

17. In claim 15, wherein the said health disorders are one or more among hypertension, hyperlipidemia, obesity, inflammatory disease, arthritis, hypercholesterolemia, cardiovascular disease, cerebrovascular disease, arteriosclerosis, diabetes mellitus, immune dysfunction and cancer in mammals.

18. In claim 15, wherein the said composition is comprised of a variety of the naturally occurring bioactives, micronutrients and antioxidants of the said vegetable oil.

19. In claim 18, wherein the said naturally occurring unsaponifiable bioactives, micronutrients and antioxidants are present in the natural lipid matrix of the vegetable oil and the unsaponifiable fraction is concentrated as a whole and not prepared as a mixture of individually isolated constituents
